(19) United States
(12) Patent Application Publication
(10) Pub. No.: US 2008/0294020 A1
(43) Pub. Date: Nov. 27, 2008

(54) SYSTEM AND METHOD FOR PHYSLOLOGICAL DATA READINGS, TRANSMISSION AND PRESENTATION

(76) Inventor: Demetrios Sapounas, Leesburg, VA (US)

Correspondence Address: ARENT FOX LLP, 1050 CONNECTICUT AVENUE, N.W., SUITE 400, WASHINGTON, DC, 20036, US

(21) Appl. No.: 12/010,447

(22) Filed: Jan. 25, 2008

Related U.S. Application Data
(60) Provisional application No. 60/897,243, filed on Jan. 25, 2007.
(60) Provisional application No. 60/899,410, filed on Feb. 5, 2007.
(60) Provisional application No. 60/900,118, filed on Feb. 8, 2007.
(60) Provisional application No. 60/900,987, filed on Feb. 13, 2007.
(60) Provisional application No. 60/924,083, filed on Apr. 30, 2007.
(60) Provisional application No. 60/924,125, filed on May 1, 2007.
(60) Provisional application No. 61/006,094, filed on Dec. 19, 2007.
(60) Provisional application No. 61/006,095, filed on Dec. 19, 2007.
(60) Provisional application No. 61/006,097, filed on Dec. 19, 2007.
(60) Provisional application No. 61/006,099, filed on Dec. 19, 2007.
(60) Provisional application No. 61/006,100, filed on Dec. 19, 2007.
(60) Provisional application No. 61/006,098, filed on Dec. 19, 2007.

(51) Int. Cl. A61B 5/02

(52) U.S. Cl. 600/301

(57) ABSTRACT

Systems, methods, and computer program products for facilitating the reading, transmission and presentation of physiological data within a wireless body area network are disclosed. The remote collection and monitoring of a person's (e.g., patient's) physiological data and activity levels for the purposes of determining the well-being of the person, as well as making additional health status determinations based on the historical information and trends of the collected data are provided. The systems, methods, and computer program products disclosed herein, in varying embodiments, readily lend themselves to incremental component and functionality modifications, which allow for increased sensor data sources, accuracy, reliability and utility of the collected information, further solidifying the uniqueness and desirability of the systems methods and computer program products.

### CROSS-REFERENCE TO RELATED APPLICATIONS

This Application is claims the benefit of, and is related to, the following of Applicants' co-pending applications:

U.S. Provisional Application No. 60/897,243 titled “Method and System for Physiological Data Readings, Transmission, and Presentation,” filed on Jan. 25, 2007;

U.S. Provisional Application No. 60/899,410 titled “Communications and Biosensor Device,” filed on Feb. 5, 2007;

U.S. Provisional Application No. 60/900,118 titled “Body Patch for Non-Invasive Physiological Data Readings,” filed on Feb. 8, 2007;

U.S. Provisional Application No. 60/900,987 titled “Physiological Data Processing Architecture for Situation Awareness,” filed on Feb. 13, 2007;

U.S. Provisional Application No. 60/924,083, titled “Heterogeneous Data Collection and Data Mining Platform,” filed on Apr. 30, 2007;

U.S. Provisional Application No. 60/924,125 titled “Heterogeneous Data Collection and Data Mining Platform” filed on May 1, 2007;

U.S. Provisional Application No. 61/006,094, titled “Improved Communications and Biosensor Device,” filed on Dec. 19, 2007;

U.S. Provisional Application No. 61/006,095, titled “Gateway for Discrete and Continuous Monitoring of Ambient Data with Emergency Functions,” filed on Dec. 19, 2007;

U.S. Provisional Application No. 61/006,097, titled “Gateway for Discrete and Continuous Monitoring of Physiological Data,” filed on Dec. 19, 2007;

U.S. Provisional Application No. 61/006,099, titled “Method and System for Discrete and Continuous Monitoring or Physiological and Ambient Data,” filed on Dec. 19, 2007;

U.S. Provisional Application No. 61/006,100, titled “User Interface for System for Discrete and Continuous Monitoring of Physiological and Ambient Data,” filed on Dec. 19, 2007; and

U.S. Provisional Application No. 61/006,098, titled “Method and System for Data Transmission for Use with Biosensor Device or Gateway,” filed on Dec. 19, 2007; each of which is incorporated by reference herein in its entirety.

### BACKGROUND OF THE INVENTION

1. Field of the Invention

The present invention generally relates to automated systems and methods for collecting physiological data, and more particularly to wireless body area network systems, methods and computer program products for facilitating the reading, transmission and presentation of such physiological data.

2. Related Art

In today's technological environment, systems containing individual sensors with (or without) wireless transceivers are known and used for collecting and transmitting physiological (and ambient or motion) data (e.g., vital signs such as blood pressure, pulse rate, respiration), which reflect the health status or well being of a person. Such systems are commonly referred to as Wireless Body Area Networks (WBANs). The goal of WBANs, and their supporting information infrastructures, is to offer unprecedented opportunities to (remotely) monitor the state of health of the wearer of such systems, without constraining the activities of the wearer. The convergence of technologies such as low-power wireless communication standards, plug-and-play device buses, off-the-shelf development kits for low-power microcontrollers, handheld computers, electronic medical records, and the Internet have allowed WBAN technologies to come about.

One example of the use of WBANs are for elderly people and/or other individuals that need frequent monitoring and thus are living in a nursing home or other managed care facility environment. Such environments, obviously, limit the monitored individuals' ability to continue living independently (e.g., in their own homes). This is primarily because care givers may not be available to constantly monitor their physiological indicators and/or ambient factors, especially in the case of care givers who do not live in close proximity to the monitored individual. Further, the costs of nursing homes and other managed facilities have skyrocketed in recent years. With the use of WBANs, however, one or more sensors of differing types are employed to remotely and ambulatorily monitor a user's physiological indicators and/or other ambient factors (e.g., motion sensors, electrocardiograms (ECGs), electromyograms (EMGs), electro-encephalograms (EEGs)). The sensors can be located on the body as wearable apparatuses or tiny intelligent patches, integrated into clothing, or even implanted below the skin or muscles.

Further, WBAN systems typically utilize a storage device for aggregating the sensed and collected data for future access and processing, or are dependent on smart phones and similar mobile devices for collecting and then transmitting the data to a healthcare provider or a health monitoring entity.

While the above-described systems work for their respective intended purposes, the state of the art is such that they are often cumbersome to put on and operate. This is true both from the perspective of weight and size of the WBAN-related equipment, as well as because many such systems require wires for interconnecting the various components.

Further, there are currently no available methods, systems and computer program products for data monitoring and transmission, such that, when data levels fall below or rise above certain pre-defined or pre-selected parameter ranges, the monitoring and transmission occur in one of a plurality of selectable modes. There are also no currently-available methods, systems and computer program products that allow for discrete monitoring and transmission of data while the monitored parameters fall within certain pre-defined or pre-selected ranges and for continuous, near real-time monitoring and transmission of data when the monitored parameters fall outside of the pre-defined or pre-selected ranges.

Given the foregoing, what are needed are improved wireless, near-real time WBAN systems, methods and computer program products for facilitating the reading, transmission and presentation of physiological data.

### BRIEF DESCRIPTION OF THE INVENTION

Embodiments of the present invention meet the above-identified needs by providing systems, methods and computer program products for facilitating the reading, transmission and presentation of physiological data.

An advantage of some embodiments of the present invention is that these embodiments provide simple-to-put-on, lightweight sensors, thus making them ideal for everyday use, without impeding the user's normal activities.

Another advantage of embodiments of the present invention is that these embodiments are completely wireless and the sensed and collected physiological and/or ambient data are made available in near-real-time, both through a secure browser connection and on mobile devices, to service subscribers.

Another advantage of embodiments of the present invention is that the sensed and collected physiological and/or ambient data is made available in discrete intervals or in a continuous transmission mode, both through a secure browser connection and via mobile devices, to service subscribers.

Another advantage of embodiments of the present invention is that a user is able to set and change physiological indicator and/or ambient factor parameter ranges, such that a deviation from these ranges would trigger a modified (e.g., continuous, near real-time) monitoring and transmission mode.

Yet another advantage of embodiments of the present invention is that a user interface is provided, such that a user may set and change information related to the monitored individual, such as pre-programmed emergency telephone numbers, contact information in case of an emergency, and the like.

Further features and advantages of embodiments of the present invention, as well as the structure and operation of these various embodiments of the present invention, are described in detail below with reference to the accompanying drawings.

### BRIEF DESCRIPTION OF THE DRAWINGS

The features and advantages of embodiments of the present invention will become more apparent from the detailed description set forth below when taken in conjunction with the drawings, in which like reference numbers indicate identical or functionally similar elements. Additionally, the left-most digit of a reference number identifies the drawing in which the reference number first appears.

FIG. 1 is a block diagram of an exemplary system for facilitating the reading, transmission and presentation of physiological data according to an embodiment of the present invention.

FIG. 2 is an electronic block diagram illustrating an exemplary body patch according to an embodiment of the present invention.

FIG. 3 is a flowchart depicting the operation and data flow of a body patch according to an exemplary embodiment of the present invention.

FIG. 4 is an electronic block diagram illustrating an exemplary body-wearable gateway device according to an embodiment of the present invention.

FIG. 5 is a flowchart depicting operation and data flow of a body-wearable gateway device according to an embodiment of the present invention.

FIG. 6 is a block diagram of an exemplary data center network architecture according to an embodiment of the present invention.

FIG. 7 is a block diagram of an exemplary computer system useful for implementing embodiments of the present invention.

FIG. 8 is a flowchart depicting operation and data flow of a data center according to an embodiment of the present invention.

FIG. 9 is a flowchart depicting operation and data flow of a data center, from a user's perspective, according to an embodiment of the present invention.

FIG. 10 is a flowchart depicting operation and data flow of a data center, from a call centre/emergency response perspective, according to an embodiment of the present invention.

### DETAILED DESCRIPTION

Embodiments of the present invention are directed to systems, methods, and computer program products for facilitating the reading, transmission and presentation of physiological data.

In an embodiment of the present invention, an integrated system for obtaining a person's physiological and/or ambient data (e.g., vital signs), through non-invasive methods, securely transmitting the information, and transforming the information into an easily-understood display is disclosed. That is, a physiological and activity data aggregation, transmission and presentation system, method, and computer program product for the purpose of monitoring a person's vital signs by the person's family members, care takers, healthcare providers and the like, through non-invasive features is disclosed. Such a system, in one embodiment, includes miniaturized physiological sensors, a gateway device, short- and long-range transceivers, software for data aggregation and transmission from multiple sensors, a data center environment with multiple server computers, and software for data storage, retrieval, manipulation, analysis, display, and transmission to an end user viewing device via, for example, the Internet. This disclosed system may be completely wireless and present the data to end users on a near-real-time basis. Furthermore, the system components placed on a person's body may be small and lightweight, so that these components do not interfere with normal daily activities. Finally, the gateway device offers an alert button for emergency two-way voice communication.

In one embodiment, the method and computer program product perform the steps of obtaining physiological data from the sensors, processing the data, encrypting the data, and then transmitting the data to the gateway device. That gateway aggregates the physiological data from the sensor sources and forwards the data to the data center. At the data center, the data is processed, analyzed, and transformed into easily understood, real-time status and historical trend displays. These displays are made available through a secure web interface for display, for example, on personal computers and mobile devices.

Embodiments of the present invention will now be described in more detail herein in terms of the above exemplary context. This description is for convenience only and is not intended to limit the application of embodiments of the present invention. In fact, after reading the following description, it will be apparent to those skilled in the relevant art(s) how to implement embodiments of the following invention in alternative ways.

The terms “person,” “patient,” “subject,” “user,” “subscriber,” “client,” “wearer,” “being,” and/or the plural form of these terms are sometimes used interchangeably herein to refer to those person(s) or other living being(s) from whom physiological data are being collected (or, in some cases, the safety and medical personnel and professionals entrusted with their well being), and thus would benefit from the system, method, and computer program products that embodiments of the present invention provide for facilitating the reading, transmission, and presentation of physiological data of persons or other living beings.

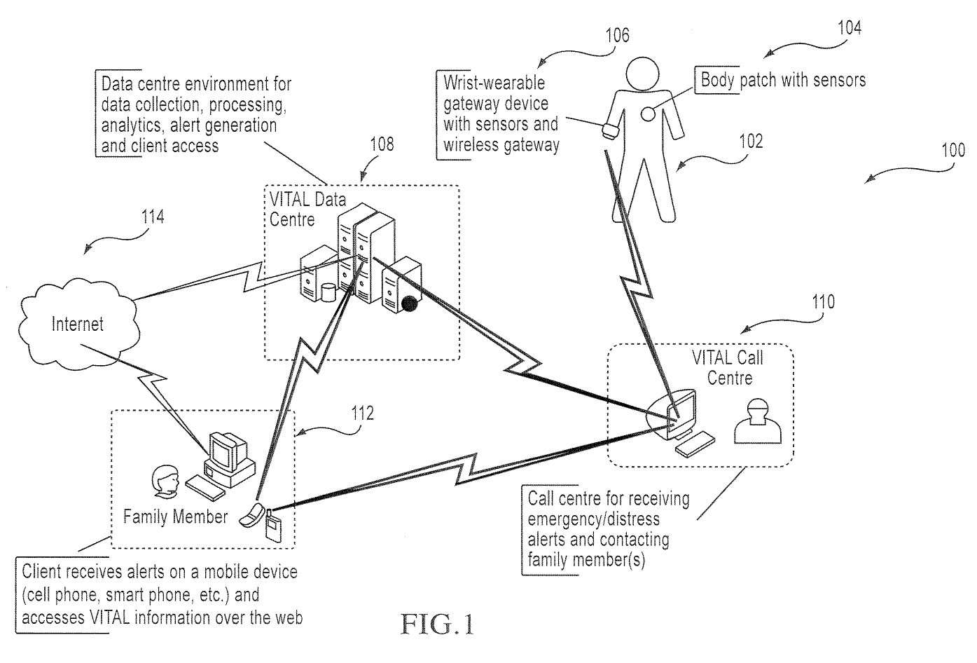

Referring to FIG. 1, a block diagram illustrating an exemplary WBAN system 100 for facilitating the reading, transmission, and presentation of physiological data, according to an embodiment of the present invention, is shown.

WBAN system 100, in one exemplary embodiment, includes a person 102 wearing a simple-to-put-on, lightweight sensor 104 attached to their body, along with a body-wearable gateway device (BWGD) 106.

In one embodiment, sensor 104 is an adhesive patch integrating several miniaturized physiological sensors, which is attached to the body. Patch 104 includes a microprocessor, a short-range wireless transceiver, and a miniaturized power supply onto a single board. The sensors obtain vital sign physiological data, which can then be processed, encrypted, and aggregated by the microprocessor for transmission by the transceiver to the gateway at pre-determined intervals.

In one embodiment, BWGD 106 is a wrist-wearable device integrating several other sensors, a microprocessor, a short-range wireless transceiver, a long-range wireless transceiver, and a power supply. BWGD 106 processes and encrypts its sensor data, then aggregates this data with the incoming radio frequency (RF) patch 104-supplied data. The microprocessor packages the aggregated data, for example, for burst transmission through the long-range transceiver at pre-determined or pre-selected intervals.

In one embodiment, BWGD 106 is in wireless communications with a data center 108. As will be appreciated by those skilled in the relevant art(s) after reading the description herein, data center 108 may be an environment of one or more networked sets of servers and communication devices operated by an entity on a per-use, subscription, or other basis for receiving and transmitting communications, processing and analyzing physiological and activity data of one or more persons 102, defining a presentation layer for data distribution to subscribers, plus managing subscriber memberships and communications.

In one embodiment, BWGD 106 is also in wireless communications with a call center 110, where a live operator may respond to the activation (e.g., the depressing) of an alert button, by the person 102 wearing device 106. This activation may be used, for example, for emergency two-way voice communication between the person 102 and personnel at the call center 110.

In one embodiment, data center 108 is in communication with a subscriber 112, who may be a family member, caretaker, medical services provider, health care provider, or the like 102. Such communications may be through a wide or local area network (WAN or LAN) running a secure communications protocol (e.g., secure sockets layer (SSL)) or the global Internet 114 using a secure web interface (e.g., Hypertext Transfer Protocol Secure (HTTPS)) for display on a personal computer or other device belonging to subscriber 112. In an alternate embodiment, such communications may be through wireless communications to a mobile device (e.g., mobile telephone or the like) belonging to subscriber 112. As will be appreciated by one skilled in the relevant art(s), subscriber 112 may receive and interface with data from data center 108 using any processing device, including, but not limited to, a desktop computer, laptop, palmtop, workstation, set-top box, mobile telephone, personal data assistant (PDA), or the like.

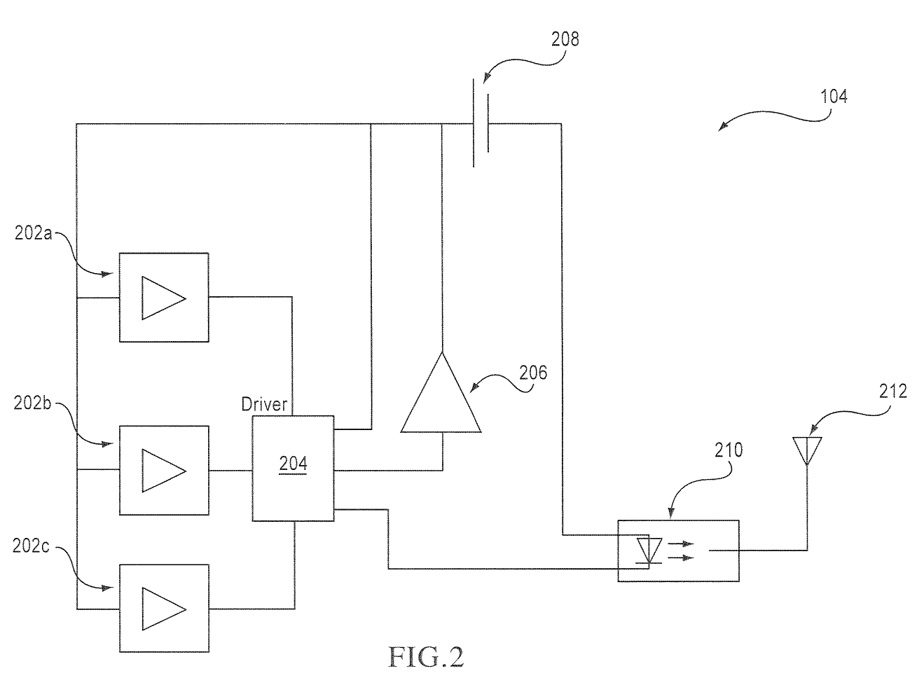

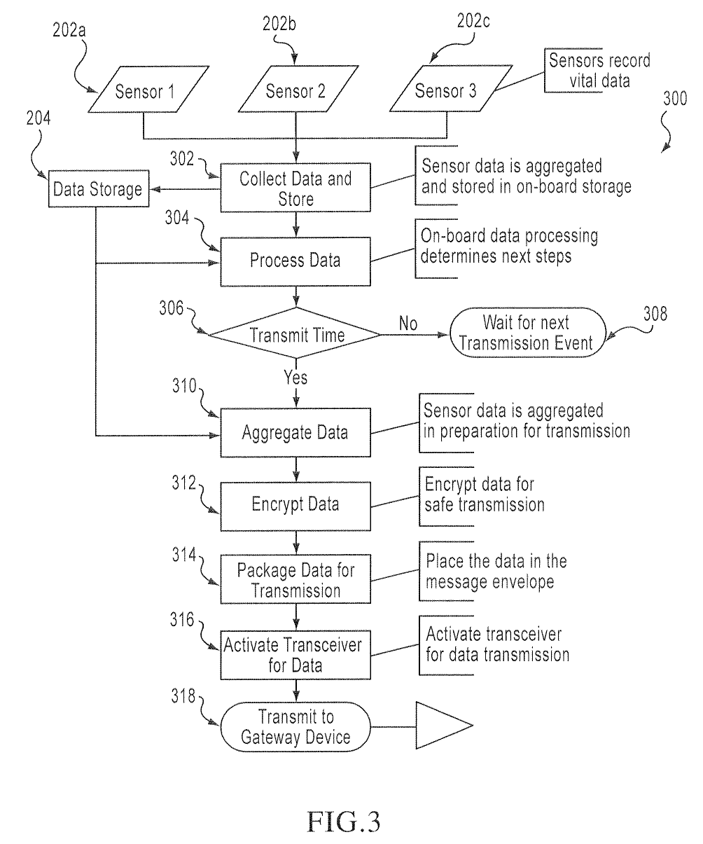

Referring to FIG. 2, an electronic block diagram of body patch 104 is shown according to an embodiment of the present invention. In such an embodiment, patch 104 comprises three sensors 202a, 202b and 202c, a microprocessor 204 with memory, an amplifier 206, a power supply 208, and a transceiver 210 with an antenna 212. Patch 104 is described in more detail in co-pending U.S. Provisional Application No. 60/900,118 titled “Body Patch for Non-Invasive Physiological Data Readings,” filed on Feb. 8, 2007.

Referring to FIG. 3, a flowchart depicting exemplary operation and data flow 300 of the patch 104 of FIG. 1 according to an embodiment of the present invention is shown. In this embodiment, the physiological and/or ambient data read by sensors 202a, 202b and 202c contained within patch 104 is collected and stored in the internal storage of microprocessor 204 in a step 302. In step 304, the data are processed for on-patch analysis. In a step 306, processor 204 determines whether to wait in a step 308 until it is time to transmit, or if it is time to initiate a transmission to BWGD 106. If step 306 determines that it is time to transmit, the transmission preparation process begins. Thus, in step 310, readings from the sensors 202a-c are aggregated and compressed.

In a step 312, the data is encrypted in preparation for transmission. Next, in step 314, the data are packaged into a message, according to the (short-range) transmission protocol being employed. Any number of protocols may be used, the majority of which specify an operating frequency range. Other protocols may operate on a single frequency. In alternate embodiments, transmission protocols may include ZigBee (802.15.4), Cellular (CDMA, TDMA, GSM and others), Wireless (802.11a/b/g/n), Wi-Fi (802.11 p), ANT, Bluetooth (802.15.1), or custom wireless protocols working in available frequencies. In step 316, transceiver 210 is activated. Finally, in step 318, a burst transmission of data from patch 104 to BWGD 106 occurs, and transceiver 210 is then deactivated until the next transmission event (e.g., until data flow 300 is repeated).

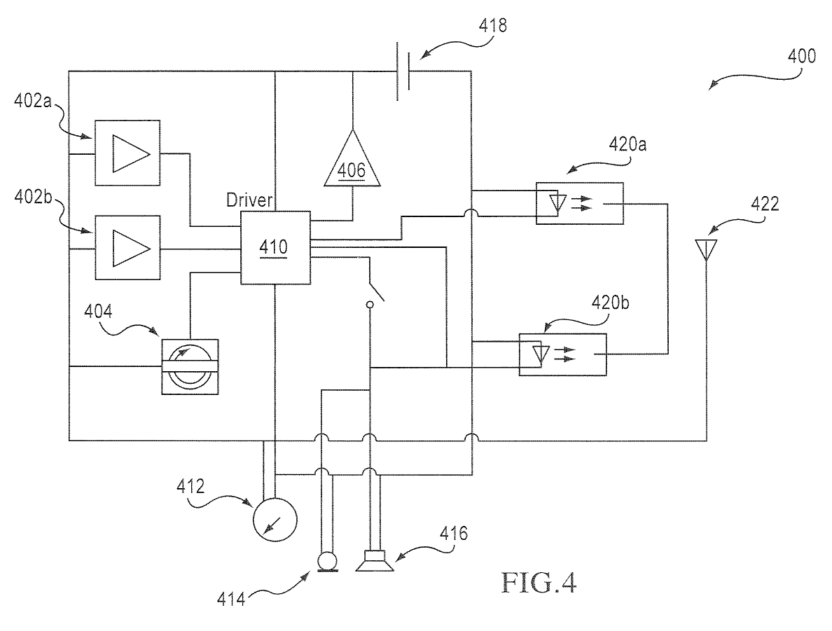

Referring to FIG. 4, an electronic block diagram illustrating an exemplary body-wearable gateway device (BWGD) 106 according to an embodiment of the present invention is shown. In this embodiment, BWGD device 106 comprises two sensors 402a and 402b, a gyroscope/accelerometer 404, an amplifier 406, a microprocessor with memory 410, an alert switch 412, a microphone 414, a speaker 416, a power supply 418, a short-range wireless transceiver 420a, a long-range wireless transceiver 420b, and an antenna 422. BWGD device 106 is described in more detail in co-pending U.S. Provisional Application No. 60/899,410 titled “Communications and Biosensor Device,” filed on Feb. 5, 2007.

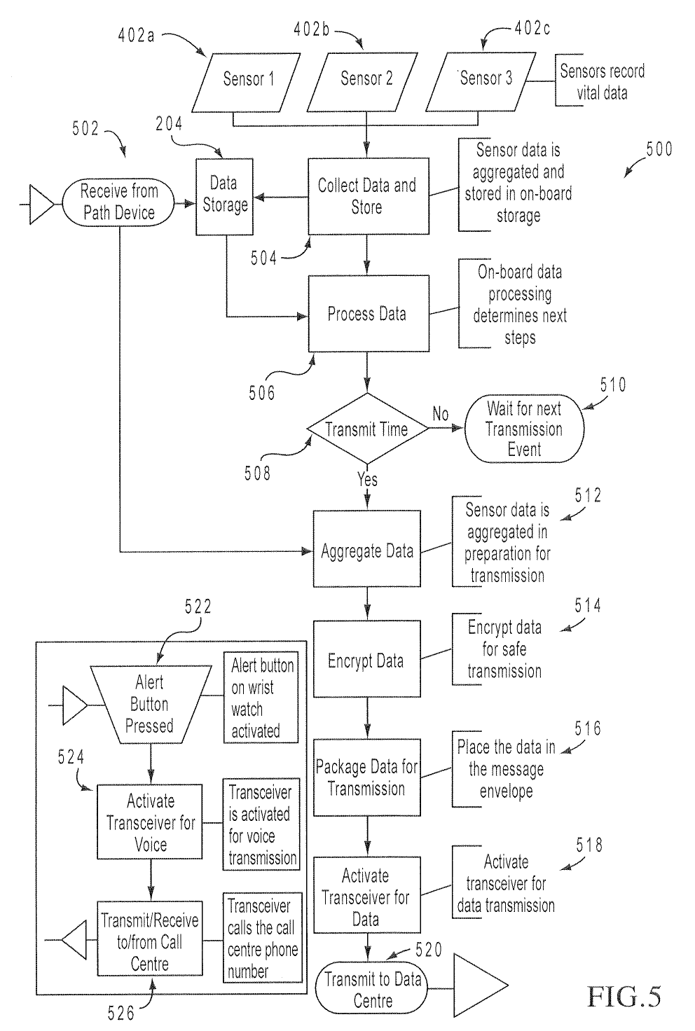

Referring to FIG. 5, a flowchart depicting operation and data flow 500 of body-wearable gateway device (BWGD) 106 in accordance with an embodiment of the present invention is shown. As will be appreciated by one skilled in the relevant art(s) after reading the description herein, data flow 500 comprises two distinct data flows—one occurring automatically as part of operations of the system 100 of FIG. 1 and the other is initiated by system wearer 102 of FIG. 1, indicating an alert condition as described below.

In one embodiment, under normal operating conditions, as shown in FIG. 5, there are two sources of data within flow 500—one from body patch 104 FIG. 1 and one from sensors 402a-c (FIG. 4) integrated within gateway device 106. In this embodiment, data are received from patch 104 (FIG. 1) via short-range transceiver 420a (FIG. 4) in step 502, and data are received from sensors 402a-c in step 504. Received data is collected and placed in internal storage on microprocessor 410. In step 506, microprocessor 410 processes the data to determine patterns and compress the data. In step 508, it is determined if it is time to initiate a scheduled transmission. If not, data flow 500 waits until it is transmission time.

In step 512, the data (including the data from the body patch 104 and gateway device 106 of FIG. 1) are aggregated in preparation for transmission. In step 514, the data are compressed and encrypted for security purposes. In step 516, a transmission message is constructed, including identifying information, destination, transmission type, and other pertinent information, according to the long-range transmission protocol being employed, as will be appreciated by those skilled in the relevant art(s). Any number of protocols may be used, the majority of which specify an operating frequency range. Other protocols may operate on a single frequency. In alternate embodiments, transmission protocols may include ZigBee (802.15.4), Cellular (CDMA, TDMA, GSM and others), Wireless (802.11a/b/g/n), Wi-Fi (802.11 p), ANT, Bluetooth (802.15.1), or custom wireless protocols working in available frequencies. In step 518, long-range transceiver 420b (FIG. 4) is activated, and as soon as a network connection is established, in step 520, the message is transmitted to data center 108 (FIG. 1) for analysis, further processing, and eventual presentation to subscribers 112 (FIG. 1).

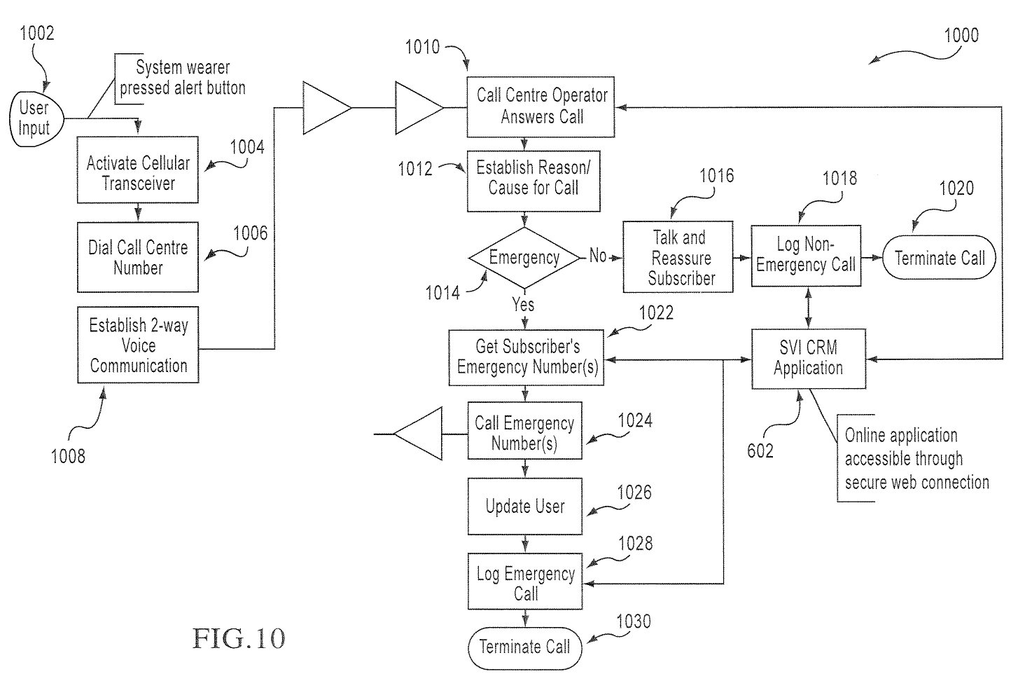

In an alternate mode of operation of data flow 500 of FIG. 5, an alert condition is initiated by wearer 102 of gateway device 106 as shown in FIG. 1. An alert condition is usually indicative of a situation requiring immediate attention by a human. Thus, in step 522, data flow is initiated by wearer 102 (FIG. 1) pressing alert button (which triggers alert switch 412 of FIG. 4) on gateway device 106 (FIG. 1). Consequently, long-range transceiver 420b (FIG. 4) is activated (in step 524) and, in one embodiment, gateway device 106 (FIG. 1) causes communications to be initiated with call center 110 of FIG. 1 (e.g., by dialing a telephone number via cellular communications), thus initiating two-way voice communication between wearer 102 (FIG. 1) and personnel at the call center 110 of FIG. 1 (in step 526). Under these circumstances wearer 102 (FIG. 1) describes the alert condition and personnel at call center 110 (FIG. 1) can take follow-on actions, which may include notifying emergency contacts of wearer 102 of FIG. 1 (e.g., subscribers 112 of FIG. 1) or contacting first responders or other emergency personnel. (See also FIG. 10 and accompanying text.)

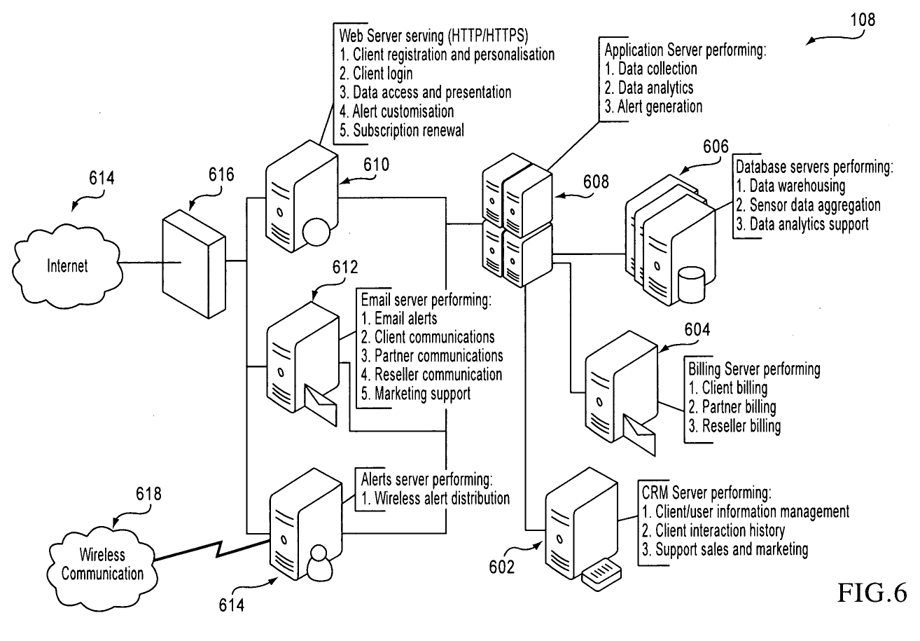

Referring to FIG. 6, a block diagram of an exemplary data center 108 network architecture according to an embodiment of the present invention is shown. In such an embodiment, as will be appreciated by those skilled in the relevant art(s), data center 108 (FIG. 1) is equipped to receive the physiological and activity data of one or more persons 102 (FIG. 1), and then process, analyze and transform such data into easily understood, real-time status and historical trend displays for presentation to one or more users (e.g., subscribers 112 of FIG. 1). In one embodiment, the components of data center 108 (FIG. 1) are connected and communicated via a wide or local area network (WAN or LAN) running a secure communications protocol (e.g., secure sockets layer (SSL)) that support data analytics and online operations, including customer service, client communications, billing, and customer relationship functions.

More specifically, in one embodiment, data center 108 (FIG. 1) includes a Customer Relationship Management (CRM) server 602, which manages information acquired from sales, marketing, customer service, and support, for example, such as user information and interaction history. A billing server 604 supports user, subscriber and reseller billing information. One or more database servers 606 perform system data warehousing, sensor and data aggregation, and data analytics support. One or more application servers 608 assemble, deploy and maintain data collection across data center 108 (FIG. 1) by facilitating alert generation and data collection and analytics. A web server 610 runs a Web site which sends out web pages in response to Hypertext Transfer Protocol (HTTP/HTTPS) requests from remote browsers (e.g., subscribers 112 of WBAN system 100 shown in FIG. 1). That is, server 610 provides the graphical user interface (GUI) to users of the system 100 (FIG. 1) in the form of Web pages. These Web pages sent to the subscriber's personal computers may result in GUI screens being displayed. Such pages may include client registration, GUI customization, data access and presentation, alert customization, subscription management, and subscription renewal pages.

In one embodiment, the connection from web server 610 to the Internet is via a firewall 616. Firewall 616 serves as the connection and separation between the WAN/LAN, which includes the plurality of system elements (e.g., servers 602-614) “inside” of the data center 108 (FIG. 1), and the global Internet 114 (FIG. 1) “outside” of data center 108 (FIG. 1). Generally speaking, firewalls are well known in the relevant art(s) and are dedicated gateway machines with special security precaution software. Firewalls are typically used, for example, to service Internet connections and dial-in lines and protect the cluster of more loosely administered network elements hidden behind it from external invasion.

Data center 108 (FIG. 1) also includes an email server 612 which handles electronic mail communications, such as email alerts and subscriber, user, and reseller and marketing personnel email communications. Data center 108 (FIG. 1) also includes an alert server 614, which is capable of communicating to subscribers 112 (FIG. 1) via one or more (long-range) wireless communications infrastructure 618 via one or more wireless communications protocols.

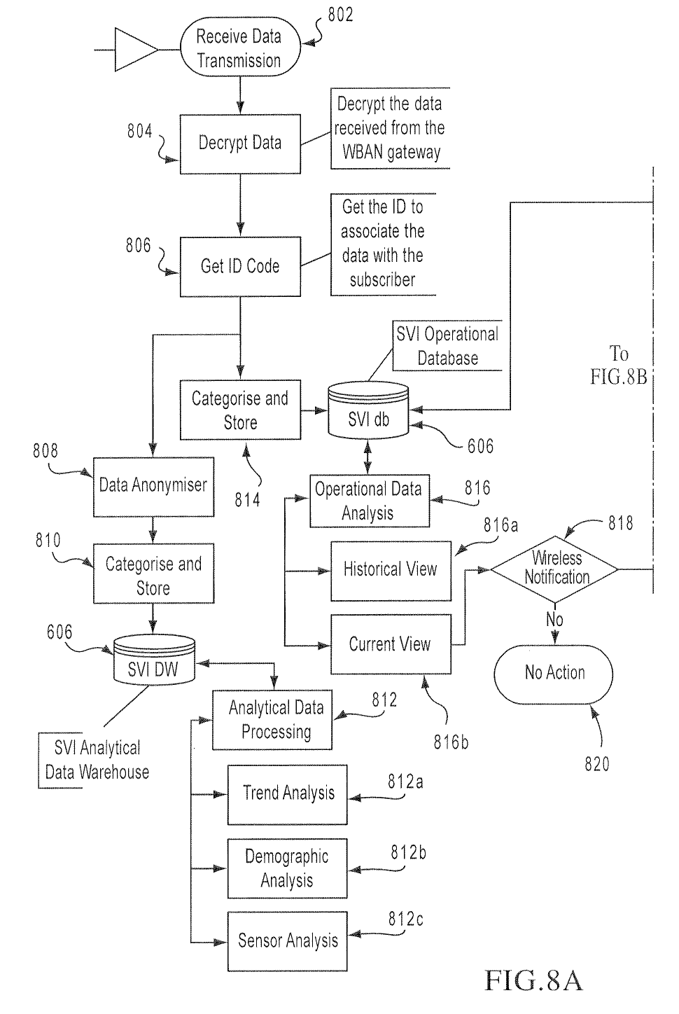

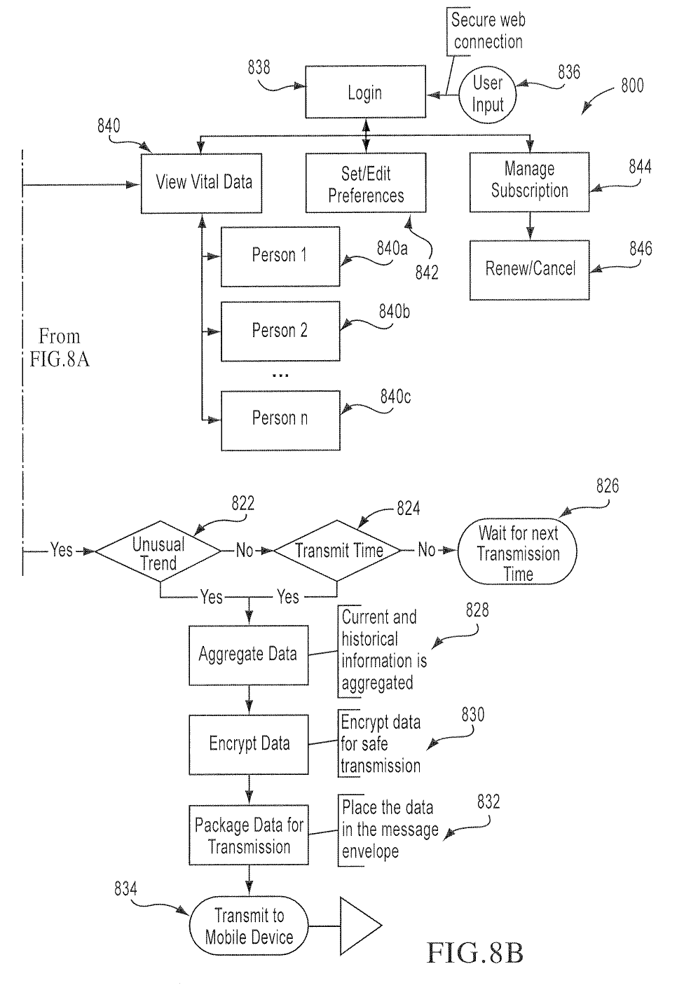

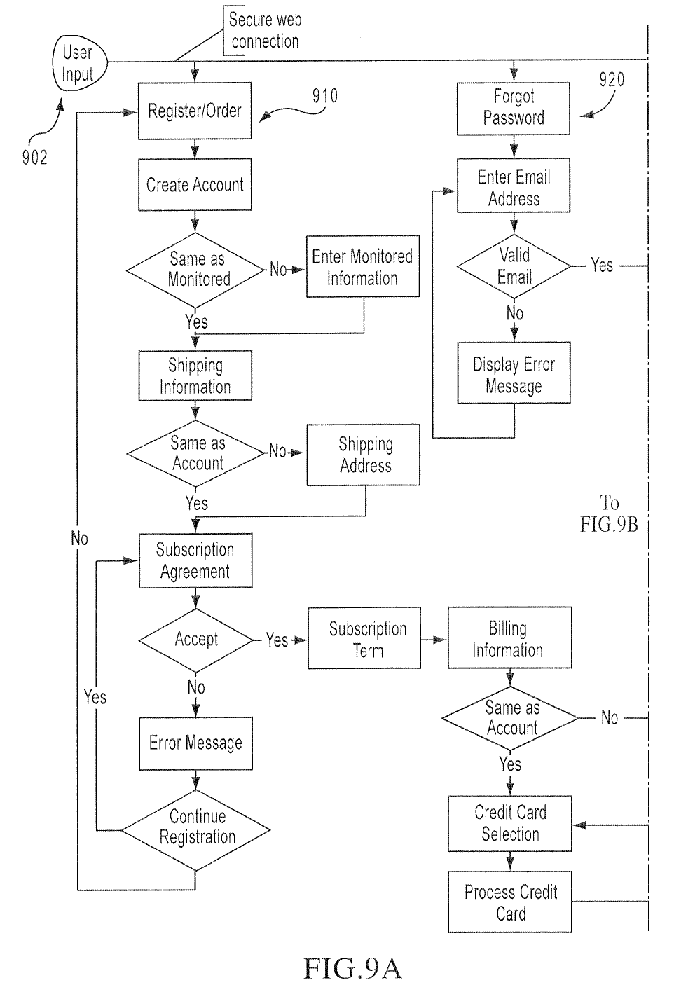

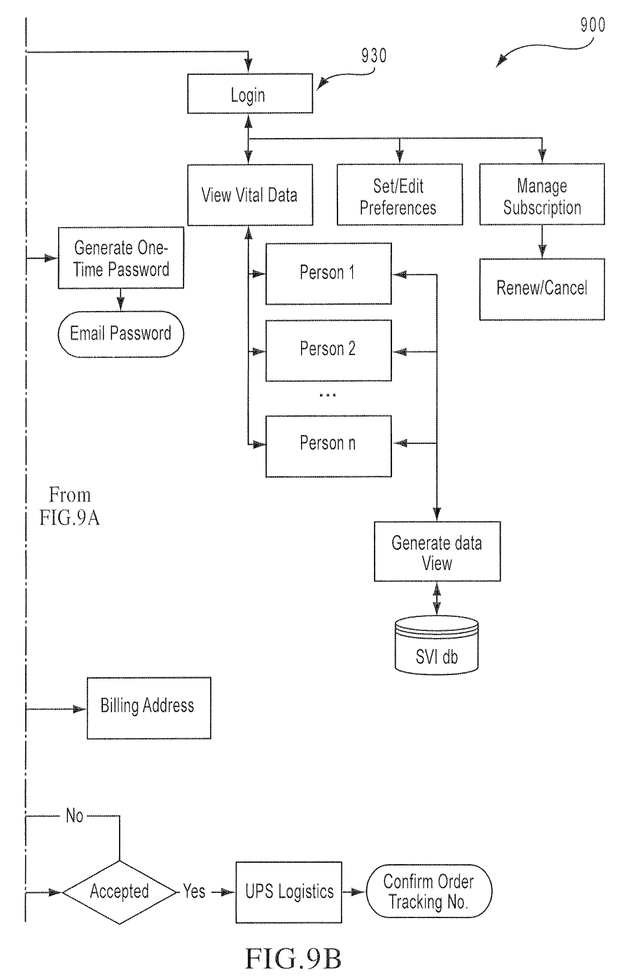

Embodiments of the present invention—e.g., system 100 (FIG. 1), the methods 300 (FIG. 3), 400 (FIG. 4), 800 (FIG. 8), 900 (FIG. 9) and 1000 (FIG. 10) for facilitating the reading, transmission and presentation of physiological data of embodiments of the present invention, or any part(s) or function(s) thereof—may be implemented using hardware, software or a combination thereof and may be implemented in one or more computer systems or other processing systems. However, the manipulations performed in accordance with embodiments of the present invention are often referred to herein in terms, such as adding or comparing, which are commonly associated with mental operations performed by a human operator. No such capability of a human operator is necessary, or desirable in most cases, in any of the operations described herein which form part of embodiments of the present invention. Rather, the operations are machine operations. Useful machines for performing the operation of embodiments of the present invention include general purpose digital computers or similar devices.

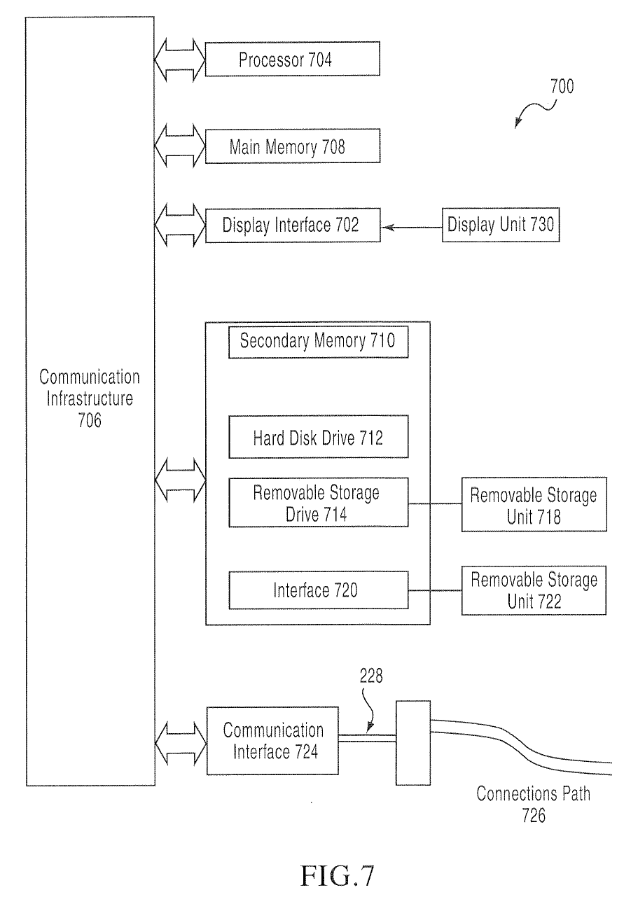

In fact, in one embodiment, the invention is directed toward one or more computer systems capable of carrying out the functionality described herein. An example of a computer system 700 is shown in FIG. 7.

The computer system 700 includes one or more processors, such as processor 704. The processor 704 is connected to a communication infrastructure 706 (e.g., a communications bus, cross-over bar, or network). Various software embodiments are described in terms of this exemplary computer system. After reading this description, it will become apparent to a person skilled in the relevant art(s) how to implement the invention using other computer systems and/or architectures.

Computer system 700 can include a display interface 702 that forwards graphics, text, and other data from the communication infrastructure 706 (or from a frame buffer not shown) for display on the display unit 730.

Computer system 700 also includes a main memory 708, preferably random access memory (RAM), and may also include a secondary memory 710. The secondary memory 710 may include, for example, a hard disk drive 712 and/or a removable storage drive 714, representing a floppy disk drive, a magnetic tape drive, an optical disk drive, etc. The removable storage drive 714 reads from and/or writes to a removable storage unit 718 in a well known manner. Removable storage unit 718 represents a floppy disk, magnetic tape, optical disk, etc. which is read by and written to by removable storage drive 714. As will be appreciated, the removable storage unit 718 includes a computer usable storage medium having stored therein computer software and/or data.

In alternative embodiments, secondary memory 710 may include other similar devices for allowing computer programs or other instructions to be loaded into computer system 700. Such devices may include, for example, a removable storage unit 722 and an interface 720. Examples of such may include a program cartridge and cartridge interface (such as that found in video game devices), a removable memory chip (such as an erasable programmable read only memory (EPROM), or programmable read only memory (PROM)) and associated socket, and other removable storage units 722 and interfaces 720, which allow software and data to be transferred from the removable storage unit 722 to computer system 700.

Computer system 700 may also include a communications interface 724. Communications interface 724 allows software and data to be transferred between computer system 700 and external devices. Examples of communications interface 724 may include a modem, a network interface (such as an Ethernet card), a communications port, a Personal Computer Memory Card International Association (PCMCIA) slot and card, etc. Software and data transferred via communications interface 724 are in the form of signals 728 which may be electronic, electromagnetic, optical or other signals capable of being received by communications interface 724. These signals 728 are provided to communications interface 724 via a communications path (e.g., channel) 726. This channel 726 carries signals 728 and may be implemented using wire or cable, fiber optics, a telephone line, a cellular link, an radio frequency (RF) link and other communications channels.

In this document, the terms “computer program medium” and “computer usable medium” are used to generally refer to media such as removable storage drive 714, a hard disk installed in hard disk drive 712, and signals 728. These computer program products provide software to computer system 700. The invention is directed to such computer program products.

Computer programs (also referred to as computer control logic) are stored in main memory 708 and/or secondary memory 710. Computer programs may also be received via communications interface 724. Such computer programs, when executed, enable the computer system 700 to perform the features of the present invention, as discussed herein. In particular, the computer programs, when executed, enable the processor 704 to perform the features of the present invention. Accordingly, such computer programs represent controllers of the computer system 700.

In an embodiment where the invention is implemented using software, the software may be stored in a computer program product and loaded into computer system 700 using removable storage drive 714, hard drive 712 or communications interface 724. The control logic (software), when executed by the processor 704, causes the processor 704 to perform the functions of the invention as described herein.

In another embodiment, the invention is implemented primarily in hardware using, for example, hardware components such as application specific integrated circuits (ASICs). Implementation of the hardware state machine so as to perform the functions described herein will be apparent to persons skilled in the relevant art(s). In yet another embodiment, the invention is implemented using a combination of both hardware and software.

Referring to FIG. 8, a flowchart depicting operation and data flow 800 of data center 108 (FIG. 1) in accordance with an embodiment of the present invention is shown. In such an embodiment, there are at least two possible data flows—one initiated by a data transmission receipt and the other by a user login to the online system.

In one embodiment, where transmission of data from gateway device 106 occurs, the transmission message is received through the wireless (e.g., cellular) network and/or through the Internet in step 802. Upon receipt, in step 804, the message is decrypted. In step 806, identifying information is extracted from the message, such that the user 102 is identifiable, and thus the ability to build data associations and determine further processing of the data is possible. As will be appreciated by those skilled in the relevant art(s), system 100 (FIG. 1) assures that this association can only be accomplished at data center 108 (FIG. 1), in order to ensure security and patient privacy.

At this point data flow 800 takes two separate pathways for two different treatments of the data.

In step 808, the data is made anonymous and all references and associations to the user (e.g., patient identifiable information) are removed, and the data retain only demographic and sensor reading information. Next, in step 810, the data are stored in a data warehouse by database server 606 and used for analytical processing. In alternate embodiments, such data may be analyzed using, for example, proprietary algorithms belonging to the entity operating data center 108 (FIG. 1) and/or off-the-shelf (e.g., OLAP) analytical processing software in step 812. In such embodiments, the analysis that can be performed on the data includes: (a) Trend Analysis (step 812a)—to provide information on how physiological readings change over time as impacted by wearer 102 activity, medications, and other influences; (b) Demographic Analysis (step 812b)—to provide information on how physiological readings may be impacted by the population demographics and identify useful patterns that may be used in providing care; and (c)

Sensor Analysis (step 812c)—to provide information on how sensor readings may provide evidence of events, thus potentially leading to prevention methods. As will be appreciated by those skilled in the relevant art(s) after reading the description herein, other analytical functions and capabilities can also be made available, as well as custom analytics developed by the entity operating WBAN system 100 (FIG. 1) and its users, through several automated methods.

The second pathway, after step 806, is the portion of data flow 800 that follows a set of steps to address the needs and requirements of the clients 112 (FIG. 1). In step 814, the data are categorized based on source, demographics, and other parameters, and stored in a client database by database server 606 so results can be accessed by clients 112 (FIG. 1). In step 816, the data are also analyzed to extract the information clients require and make it possible to present that information in a succinct and easily understood manner. Furthermore, the data are also organized in historical and current (point in time) views to be presented to clients in steps 816a-b, respectively.

At this point, the data flow 800 follows a number of operational steps, as determined by profiles and dissemination requirements set by clients 112 (FIG. 1). A client 112 (FIG. 1) may have opted for wireless transmission of the data as determined by step 818. If that is not the case, no further action is taken (as indicated by step 820). If a client 112 (FIG. 1), however, has requested wireless transmission, the data is prepared for transmission. If the data evidences an “unusual” trend, as determined by step 822, or it is time to transmit based on a pre-determined or pre-selected schedule, as determined in step 824, the data is prepared for transmission; otherwise, data flow 800 waits until the next transmission event (as indicated by step 826).

In a step 828, the data are aggregated in preparation for transmission. In step 830, the data are compressed and encrypted for security purposes, based on the specifications of the eventual receiving device used by the client 112 (FIG. 1). In step 832, a transmission message is constructed, including identifying information, destination, transmission type, and other pertinent information, according to the (long-term) transmission protocol being employed, as will be appreciated by those skilled in the relevant art(s). In step 834, the message is transmitted to subscribers 112 (FIG. 1) on their (mobile) processing devices including, but not limited to, a computer, laptop, mobile telephone, palmtop, personal data assistant (PDA), or the like.

As stated above, within data flow 800, a client (e.g., person 102 or subscriber 112 of FIG. 1) may initiate a data transmission, as indicated in step 836. This transmission if performed, for example, through a user who accesses a secure website via a login procedure (as indicated in step 838) to obtain information or perform other actions. This procedure is commonly performed through a secure web browser connection managed by web server 610 (FIG. 6).

In one embodiment, an online user 112 (FIG. 1) can access the vital data and displays for one or more persons 102 (FIG. 2) in their subscription (as indicated by steps 840 and 840a-n.) A user may also view current data and historical trends for each person authorized in their subscription. Through this interface, a user obtains a complete update of the condition of a wearer 102 (FIG. 1), as indicated by the vital signs data collected, aggregated and transmitted by system 100 (FIG. 1).

As will be appreciated by those skilled in the relevant art(s) after reading the description herein, once connected to and authenticated by the site, a user can edit or set account preferences, which may include settings for alerts and conditions triggering them, alert and notification levels, notification preferences, notification lists, contact information, and the like (as indicated by step 842). A user can also manage their subscription (as indicated by step 844) with options for renewing or cancelling the service provided by the entity operating WBAN system 100 of FIG. 1 (as indicated by step 846).

Turning now to the data flow of users of WBAN system 100 (FIG. 1) from the perspective of a subscriber 112 (FIG. 9) and then from the perspective of wearer 102 of FIG. 1 (FIG. 10).

Referring to FIG. 9, a flowchart depicting operation and data flow 900 of a data center, from the perspective of a subscriber 112 (FIG. 1), according to an embodiment of the present invention is shown. It will be apparent to those skilled in the relevant art(s), after reading the description herein, that an online user 112 (FIG. 1) may accesses system 100 (FIG. 1) via a web browser and a secure web connection. Data flow 900 then allows a user 112 (FIG. 1), once on the home page provided by web server 610 (FIG. 6), to have the options of: creating an account (via a series of steps 910); requesting a forgotten password (via a series of steps 920); or logging into a pre-existing account (via a series of steps 930); all via user input (e.g., selection) step 902.

In one embodiment, user input step 902 proceeds to the series of steps 910, such that a user 112 (FIG. 1) can create an account on system 100 (FIG. 1). As will be apparent to those skilled in the relevant art(s) after reading the description herein, a new user may follow a series of steps 910 for establishing an account and a subscription. First, for example, the user may be asked to differentiate between the person 102 (FIG. 1) being monitored 102 (FIG. 1) and the person 112 (FIG. 1) establishing the account. If the two people are different, the monitored person's information may need to be entered. That information may include name, address, telephone number(s), age, gender, race, emergency contact information, etc. Next, shipping information may need to be entered for delivery of equipment (e.g., patch 104 and gateway device 106 of FIG. 1). After that, the subscription agreement may need to be accepted. If the agreement is not accepted, the user may have the option of either going back to the agreement and accepting it or exiting the registration process. Following agreement acceptance, the user may be asked to select a subscription duration and then enter billing and payment information. The payment information may be used for the initial shipment and subsequent payments of the subscription and patches 104 (FIG. 1). Upon verification of the payment information, the order may be processed by (overnight) courier logistics, and an order confirmation and shipment tracking number may be provided via email.

In one embodiment, user input step 902 proceeds to the series of steps 920, such that a user can retrieve (e.g., forgotten or lost) login and/or password information to access a preexisting account on system 100 (FIG. 1). As will be apparent to those skilled in the relevant art(s) after reading the description herein, the user may need to provide some identifying information, including the email address on record, a name and a telephone number. If the information is located in system 100 of FIG. 1 (e.g., CRM server 602 of FIG. 6), a one-time password may be generated and emailed to the user, along with a link for validating the password. The user may then have to go to the link included in the email, enter the one-time password, and establish a new password, to be used for subsequent access to the system.

In one embodiment, user input step 902 proceeds to the series of steps 930, such that a user 112 (FIG. 1) can login to a preexisting account on system 100 (FIG. 1). As will be apparent to those skilled in the relevant art(s) after reading the description herein, a user can login into system 100 (FIG. 1) by authenticating with the proper credentials (e.g., username and password). Following authentication, a user can edit or set account preferences, which include settings for alerts and conditions triggering them, notification preferences, list of persons and contact information for online access, normal transmissions, alerts, and other communications, as described above. A user can also manage the subscription with options for renewing or cancelling the service as described above. A user can also view the vital signs of a person 102 (FIG. 1) wearing the sensor system (i.e., patch 104 and gateway device 106 of FIG. 1) and associated with the account also as described above.

Referring to FIG. 10, a flowchart depicting operation and data flow 1000 of a data center, from the perspective of a wearer 102 (FIG. 1), according to an embodiment of the present invention is shown. In such an embodiment, wearer 102 (FIG. 1) does not need to interact with system 100 (FIG. 1), but the ability is provided to address emergency situations.

As described above with reference to FIG. 5, an alert condition may be initiated by wearer 102 (FIG. 1) of gateway device 106 (FIG. 1). An alert condition is usually indicative of a situation requiring immediate attention by a human operator. Thus, in step 1002, data flow 1000 is initiated by wearer 102 (FIG. 1) pressing alert button (which triggers alert switch 412 of FIG. 4) on gateway device 106 (FIG. 1). Consequently, long-range transmitter 420b (FIG. 4) is activated (in step 1004) and, in one embodiment, gateway device 106 (FIG. 1) causes communications to be initiated with call center 110 of FIG. 1 (e.g., by dialing a telephone number via cellular communications in step 1006), thus initiating two-way voice communication between wearer 102 (FIG. 1) and personnel at call center 110 of FIG. 1 (in steps 1008-1010). Under these circumstances wearer 102, FIG. 1) describes the alert condition, and personnel at call center 110 (FIG. 1) can take follow-on actions, which may include notifying the emergency contacts of wearer 102 (FIG. 1), first responders, or other emergency personnel (e.g., one or more subscribers 112 of FIG. 1).

In one embodiment, as a data center operator answers the telephone, the operator's computer screen displays the caller's information, and the operator starts a conversation with the caller. The conversation may include a scripted question and answer exchange, with the objective of establishing the exact reason for the call (step 1012). Once the reason is established, follow-up actions are taken. For example, if the call is determined not to be due to an emergency in step 1014, the operator converses with the caller in step 1016 and reassures the person. When the person is at ease, the operator logs the non-emergency call in CRM system 602 of FIG. 6 (in step 1018), along with a short description of what transpired, and then terminates the call (in step 1020).

If, in step 1014, the call is identified as an emergency situation, the operator uses the emergency numbers for the caller, as recorded in CRM system 602 of FIG. 6, and calls the emergency contacts until one is reached and the emergency is described (steps 1022 and 1024, respectively). The operator informs the caller of the results (in step 1026) before entering the emergency call data in CRM system 602 of FIG. 6 (in step 1028) and terminates the call (in a step 1030).

While various embodiments of the present invention have been described above, it should be understood that they have been presented by way of example, and not limitation. It will be apparent to persons skilled in the relevant art(s) that various changes in form and detail can be made therein without departing from the spirit and scope of embodiments of the present invention. Thus, embodiments of the present invention should not be limited by any of the above described exemplary embodiments, but should be defined only in accordance with the following claims and their equivalents.

In addition, it should be understood that the figures in the attachments, which highlight the structure, methodology, functionality and advantages of embodiments of the present invention, are presented for example purposes only. Embodiments of the present invention is sufficiently flexible and configurable, such that it may be implemented in ways other than that shown in the accompanying figures.

Further, the purpose of the foregoing Abstract is to enable the U.S. Patent and Trademark Office and the public generally, and especially the scientists, engineers and practitioners in the relevant art(s) who are not familiar with patent or legal terms or phraseology, to determine quickly from a cursory inspection the nature and essence of this technical disclosure. The Abstract is not intended to be limiting as to the scope of embodiments of the present invention in any way.

What is claimed is:

1. A system for facilitating the reading, transmission and presentation of physiological data, comprising:
(a) a patch capable of being attached to the body of a person, wherein the patch comprises a plurality of sensors, wherein each of the plurality of sensors is capable of measuring a physiological parameter indicative of the health status of the person;
(b) a gateway device wearable by the person, wherein said gateway device is in short-range wireless communications with the patch in order to receive a plurality of physiological parameters from the plurality of sensors; and
(c) a data center remotely located from said gateway device and capable of long-range wireless communications with the gateway device in order to receive the plurality of physiological parameters from the plurality of sensors;
wherein a user is able to remotely monitor the health status of the person by utilizing a processing device capable of wireless communications with the data center.

2. The system of claim 1, wherein the processing device capable of wireless communications with the data center is one selected from a group consisting of a desktop computer, a laptop computer, a palmtop computer, a workstation, a set-top box, a mobile telephone, and a personal data assistant.

3. The system of claim 1, wherein the gateway device comprises:
(d) a short-range transceiver in wireless communications with the patch; and
(e) a long-range transceiver in wireless communications with the data center.

4. The system of claim 3, wherein the gateway device further comprises:
(f) a plurality of gateway sensors, wherein each of the plurality of gateway sensors is capable of measuring a physiological parameter indicative of the health status of the person; and wherein each of the plurality of gateway sensors measure a physiological parameter different than the plurality of sensors located on the patch.

5. The system of claim 1, further comprising:
(d) a call center remotely located from the gateway device and capable of long-range, two-way wireless voice communications with the gateway device in order to receive indications of an alert condition.

6. The system of claim 1, wherein the data center comprises at least one server capable of analyzing at least one of the plurality of physiological parameters received from the gateway device.

7. The system of claim 6, wherein the data center further comprises at least one server capable of presenting at least one of the plurality of physiological parameters to the user utilizing the processing device.

8. The system of claim 1, wherein the gateway device is in short-range wireless communications with the patch via a wireless communications protocol selected from a group consisting of: ZigBee, Wi-Fi, ANT, and Bluetooth.

9. The system of claim 1, wherein the gateway device is in long-range wireless communications with the data center via a cellular communications protocol.

10. The system of claim 1, wherein the data center comprises an identifier feature configured to extract identifier information from the long-range wireless communications, identify the person from the identifier information, and associate data in the communication with the corresponding user.

11. The system of claim 1, wherein the data center comprises a data analysis feature that includes at least two different pathways for at least two different treatments of data within the long-range wireless communications, wherein a first pathway includes an individual analysis feature configured to associate the data with a person and analyze the data for that person, and wherein a second pathway includes an anonymous analysis feature configured to remove all patient identifiable information from the data to form anonymous data, accumulate anonymous data from a plurality of persons, and store the anonymous data for analysis.

12. A method for facilitating the reading, transmission and presentation of physiological data, the method comprising the:
(a) measuring a first plurality of physiological parameters indicative of the health status of a person by utilizing a patch capable of being attached to the body of the person, wherein the patch comprises a plurality of sensors, and wherein each of the plurality of sensors is capable of measuring a physiological parameter;
(b) transmitting, via short-range wireless communications, the first plurality of physiological parameters from the patch to a gateway device worn by the person;
(c) measuring a second plurality of physiological parameters indicative of the health status of the person by utilizing a plurality of integrated sensors located on the gateway device, wherein each of the plurality of integrated sensors is capable of measuring a physiological parameter different than the plurality of sensors located on the patch;
(d) receiving, via long-range wireless communications, the first plurality of physiological parameters and the second plurality of physiological parameters from the gateway device at a data center remotely located from the gateway device; and
(e) presenting at least one of said first plurality of physiological parameters and at least one of the second plurality of physiological parameters to a user utilizing a processing device to remotely monitor the health status of the person.

13. The method of claim 12, wherein the processing device capable of wireless communications with the data center is selected from a group consisting of a desktop computer, a laptop computer, a palmtop computer, a workstation, a set-top box, a mobile telephone, and a personal data assistant.

14. The method of claim 12, further comprising of:
(f) analyzing, at the data center, at least one of the first plurality of physiological parameters received from the gateway device; and
(g) analyzing, at the data center, at least one of the second plurality of physiological parameters received from the gateway device.

15. The method of claim 14, which includes:
(h) presenting at least one of s the first plurality of physiological parameters and at least one of the second plurality of physiological parameters to the user utilizing the processing device via wireless communications.

16. The method of claim 15, wherein presenting at least one of s the first plurality of physiological parameters and at least one of the second plurality of physiological parameters to the user utilizing the processing device via wireless communications is accomplished using a graphical user interface.

17. The method of claim 14, wherein presenting at least one of said first plurality of physiological parameters and at least one of the second plurality of physiological parameters to a user utilizing a processing device to remotely monitor the health status of the person includes:
(h) presenting at least one of the first plurality of physiological parameters and at least one of the second plurality of physiological parameters to the user utilizing said processing device via the Internet.

18. The method of claim 17, presenting at least one of s the first plurality of physiological parameters and at least one of the second plurality of physiological parameters to the user utilizing the processing device via wireless communications is accomplished using a web page.

19. The method of claim 12, further comprising:
(f) extracting identifier information from the long-range wireless communications;
(g) identifying the person from the identifier information, and
(h) associating data in the communication with the corresponding person.

20. The system of claim 12, further comprising:
separating data in the long-range wireless communication into at least two different pathways for at least two different treatments of data, wherein a first pathway includes an associating the data with a person and analyze the data for that person, and wherein a second pathway includes removing all patient identifiable information from the data to form anonymous data, accumulating anonymous data from a plurality of persons, and storing the anonymous data for analysis.

21. A computer program product comprising a computer usable medium having control logic stored therein for causing a computer to read, transmit and present physiological data, the control logic comprising:
first computer readable program code means for causing the computer to receive, via wireless communications, a first plurality of physiological parameters measured by a patch worn by a person and indicative of the health status of the person;
second computer readable program code means for causing the computer to receive, via wireless communications, a second plurality of physiological parameters measured by a gateway device worn by the person and indicative of the health status of the person;
third computer readable program code means for causing the computer to analyze at least one of the first plurality of physiological parameters and at least one of the second plurality of physiological parameters, thereby producing presentable data; and
fourth computer readable program code means for causing the computer to transmit the presentable data to a user remotely located from the person, the user utilizing a processing device to monitor the health status of the person.

22. The computer program product of claim 21, wherein the presentable data is transmitted to the user via wireless communications.

23. The computer program product of claim 22, further comprising:
fifth computer readable program code means for causing the computer to display the presentable data in the form of a graphical user interface.

24. The computer program product of claim 21, wherein the presentable data is transmitted to the user via the Internet.

25. The computer program product of claim 24, further comprising:
sixth computer readable program code means for causing the computer to display the presentable data in the form of a web page.
